(19) United States
(12) Patent Application Publication
(10) Pub. No.: US 2018/0095049 A1
(43) Pub. Date: Apr. 5, 2018

(54) HAND-HELD TEST METER WITH ANALYTICAL TEST STRIP CONTACT PRESSURE FEATURE

(71) Applicant: LifeScan Scotland Limited, Inverness (GB)

(72) Inventors: David ELDER, Inverness (GB); Jonathan NELSON, Wayne, PA (US); Alistair LONGMUIR, Inverness (GB)

(21) Appl. No.: 15/281,563

(22) Filed: Sep. 30, 2016

(51) Int. Cl. G01N 27/327; G01N 33/49; G01N 33/487

(52) U.S. Cl. CPC G01N 27/3273; G01N 2400/00; G01N 33/48785; G01N 33/49

(57) ABSTRACT

A hand-held test meter for use with an analytical test strip (such as an electrochemical-based analytical test strip) for the determination of an analyte (e.g., glucose) in a bodily fluid sample, the hand-held test meter including a housing with an analytical test strip contact pressure feature, a printed circuit board (PCB) with at least one solder bump disposed in the housing. The housing of the hand-held test meter is configured for the insertion of an analytical test strip therein. In addition, the analytical test strip contact pressure feature is configured to operatively apply pressure on the inserted analytical test strip such that at least one electrical contact of the inserted analytical test strip is operably pressed against the solder bump, thereby creating a direct operable electrical connection between the inserted analytical test strip and the solder bump of the PCB.

### BACKGROUND OF THE INVENTION

### Field of the Invention

The present invention relates, in general, to medical devices and, in particular, to test meters for use with analytical test strips and related methods.

### Description of Related Art

The determination (e.g., detection and/or concentration measurement) of an analyte in a fluid sample is of particular interest in the medical field. For example, it can be desirable to determine glucose, ketone bodies, cholesterol, lipoproteins, triglycerides, acetaminophen and/or HbA1c concentrations in a sample of a bodily fluid such as urine, blood, plasma or interstitial fluid. Such determinations can be achieved using analytical test strips along with an associated test meter.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a simplified perspective view of a hand-held test meter according to an embodiment of the present invention;

FIG. 2 is a simplified perspective exploded view of the hand-held test meter of FIG. 1 with an electrochemical-based analytical test strip inserted therein;

FIG. 3 is a simplified cross-sectional view of a portion of the hand-held-test meter of FIG. 2;

FIG. 4 is a simplified bottom perspective view of a portion of a printed circuit board (PCB) illustrating the disposition of six solder bumps thereon according to an embodiment of the present invention;

FIG. 5 is a simplified top perspective view of the portion of a PCB of FIG. 4 in operable contact with an analytical test strip (TS′);

FIG. 6 is a bottom view of the portion of the PCB of FIG. 4 with two locations where pressure is being applied (from the top) being illustrated for explanatory purposes;

FIG. 7 is a simplified bottom perspective view of a portion of another PCB illustrating the disposition of five solder bumps thereon according to another embodiment of the present invention;

FIG. 8 is a simplified top perspective cross-sectional view of the portion of the PCB of FIG. 7, taken through two solder bumps thereof, in operable contact with an analytical test strip (TS″); and

FIG. 9 is a flow diagram depicting stages in a method for employing a hand-held test meter for use with an analytical test strip in the determination of an analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, hand-held test meters for use with an analytical test strip (such as an electrochemical-based analytical test strip) for the determination of an analyte (e.g., glucose) in a bodily fluid sample according to the present invention include a housing with at least one analytical test strip contact pressure feature, a printed circuit board (PCB) with at least one solder bump, the PCB being disposed in the housing, and a micro-controller disposed on the PCB. The housing of the hand-held test meter is configured for the insertion of an analytical test strip therein. In addition, the analytical test strip contact pressure feature is configured to operatively apply pressure on an inserted analytical test strip such that the electrical contact of the inserted analytical test strip is operably pressed against the solder bump, thereby creating a direct operable electrical connection between the analytical test strip and the solder bump of the PCB.

Hand-held test meters according to the present invention are beneficial in that they include relatively few components. Moreover the analytical test strip contact pressure feature and the PCB solder (bumps) can be forms using relatively inexpensive manufacturing techniques. For example, the solder bumps can be formed directly on the PCB (e.g., on a copper trace of the PCB) during a conventional PCB electronic assembly reflow process, and are therefore very low cost.

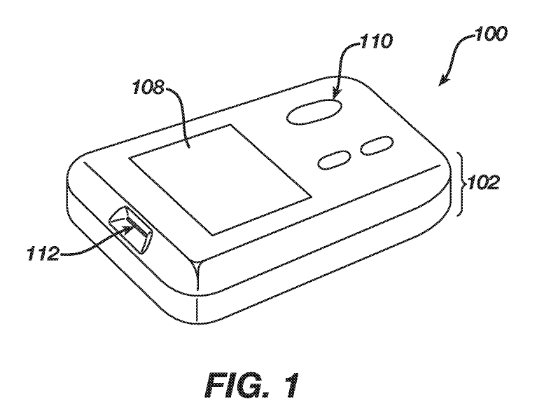

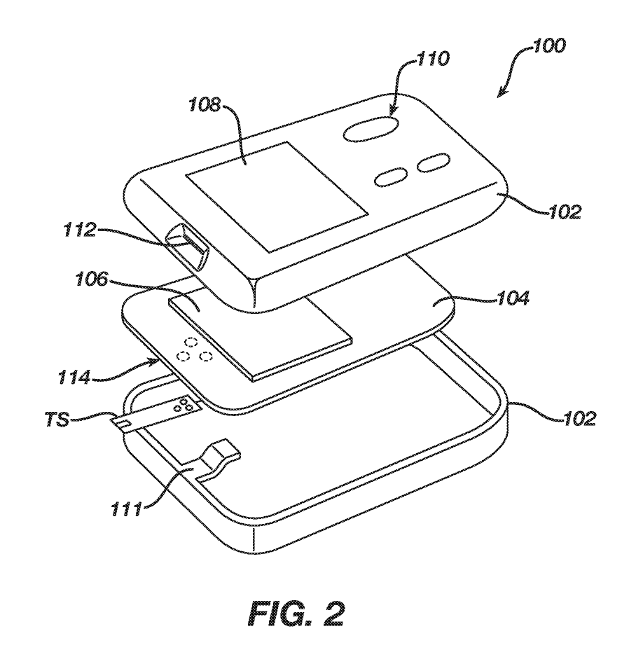

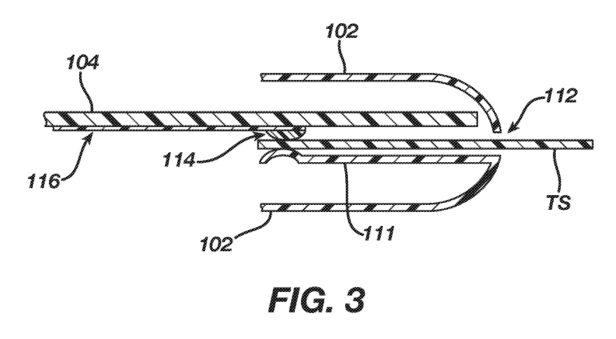

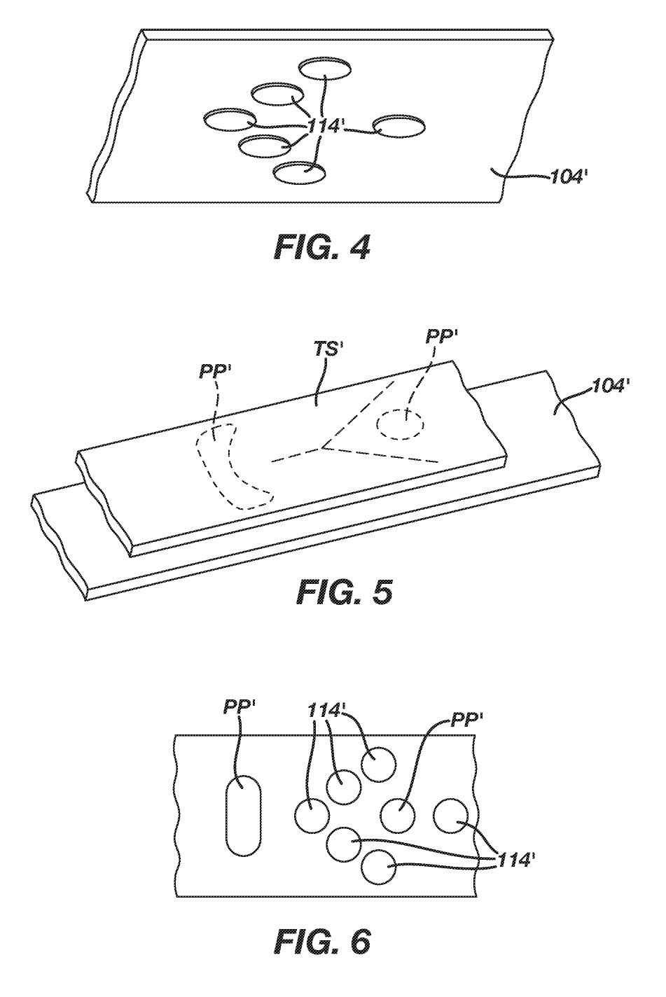

FIG. 1 is a simplified perspective view of a hand-held test meter 100 according to an embodiment of the present invention. FIG. 2 is a simplified perspective exploded view hand-held test meter 100 of FIG. 1 with an electrochemical-based analytical test strip TS inserted therein. FIG. 3 is a simplified cross-sectional view of a portion of hand-held-test meter 100. FIG. 4 is a simplified perspective view of the analytical test strip pressure feature included in hand-held test meter 100.

FIG. 4 is a simplified bottom perspective view of a portion of a PCB 104′ illustrating the disposition of six solder bumps thereon according to an embodiment of the present invention. FIG. 5 is a simplified top perspective view of the portion of the PCB of FIG. 4 in operable contact with an analytical test strip (TS′) with the areas enclosed by small dashed line representing where pressure is applied by an analytical test strip pressure feature (not depicted) and the large dashed lines representing where the analytical test strip will be slightly bent (i.e., folded or deflected by the force exerted by the analytical test strip pressure feature). FIG. 6 is a bottom view of the portion of the PCB of FIG. 4 with two locations where pressure is being applied (from the top) being illustrated for explanatory purposes.

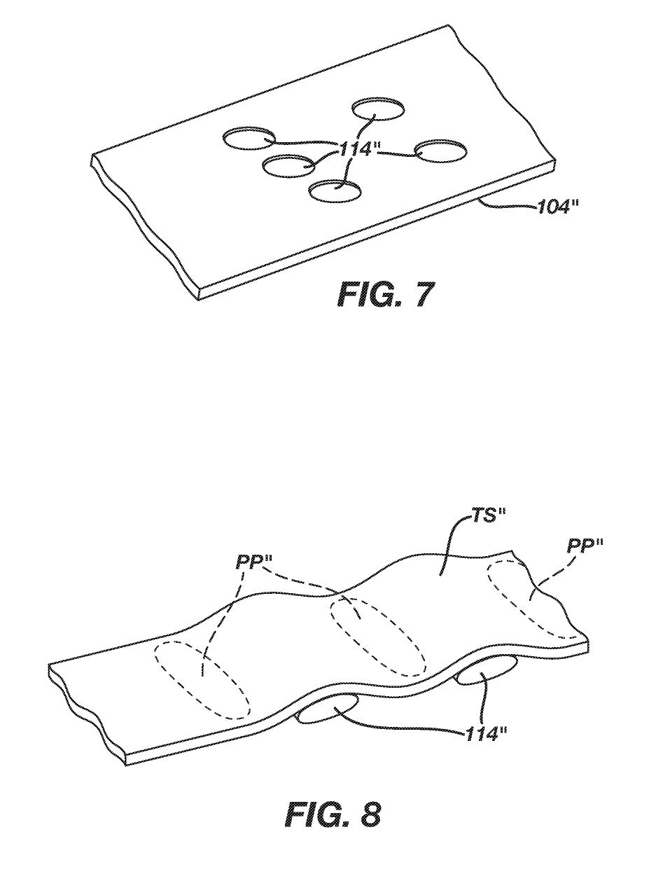

FIG. 7 is a simplified bottom perspective view of a portion of another PCB 800 illustrating the disposition of five solder bumps thereon according to another embodiment of the present invention. FIG. 8 is a simplified cross-sectional top perspective view of the portion of the PCB of FIG. 7 in operable contact with an analytical test strip (TS″) with the areas enclosed by small dashed lines indicating where pressure is applied by the analytical test strip pressure feature. The cross-sectional view of FIG. 8 is longitudinal in the perspective of FIG. 7 and through two solder bumps 114″ and is simplified to illustrate the folding of analytical test strip TS″ about solder bumps 114″.

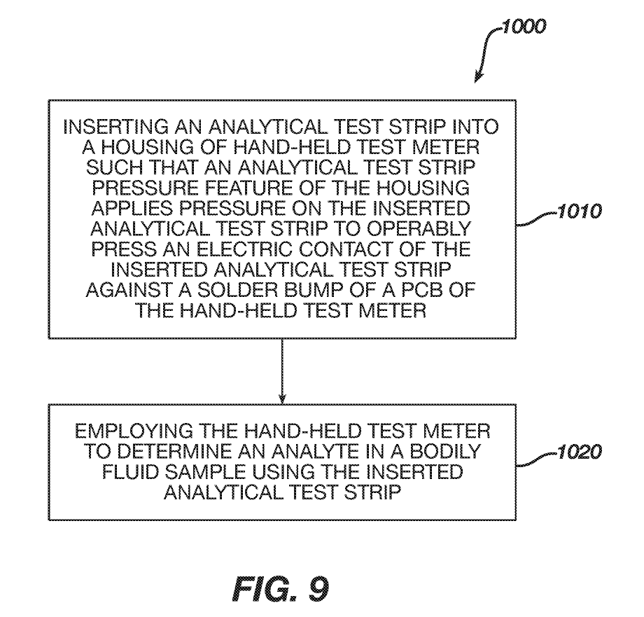

FIG. 9 is a flow diagram depicting stages in a method for employing a hand-held test meter for use with an analytical test strip (e.g., an electrochemical-based analytical test strip configured for the determination of glucose in a whole blood sample) in the determination of an analyte in a bodily fluid sample according to an embodiment of the present invention.

Referring to FIGS. 1 through 3, hand-held test meter 100 for the determination of an analyte in a bodily fluid sample according to an embodiment of the present invention includes a housing 102 (shown exploded into upper and lower portions in FIG. 2), a PCB 104, a micro-controller 106 disposed on PCB 104, a display 108 and a key pad 110.

Housing 102 includes at least one analytical test strip pressure feature 111 and a strip port opening 112 configured for the operable insertion of an analytical test strip therethrough and into housing 102. Housing 102 can be formed of any suitable material including, for example, polycarbonate materials, thermo-plastic polymeric materials, glass-reinforced thermoplastic polymeric materials, acrylonitrile butadiene styrene (ABS) materials, liquid-crystal polymer, polymeric materials in general, and combinations thereof.

The analytical test strip pressure feature employed in embodiments of the present invention can be, for example, a “sprung” analytical test strip pressure plate feature formed integrally with the meter housing (as depicted in, for example, FIGS. 2 and 3) or a separate component of the hand-held test meter attached, for example to the housing using convention methods such as a fastener(s). In such an embodiment, the analytical test strip pressure plate and its beneficial functions are extremely inexpensive since hand-held test meters typically include housing. The term “sprung” refers to analytical test strip pressure features, including analytical test strip pressure plate features, which exhibit a spring-like function by providing a force when deflected with the force generally increasing as the deflection increases. In embodiments of the present invention, such defection is created by the insertion of an analytical test strip into the hand-held test meter. The analytical test strip pressure feature can be made of any suitable material including, for example, thermoplastic materials, metals, and flexible woven materials such as a flexible fiberglass.

PCB 104 is disposed within housing 102 and includes a plurality of solder bumps 114 on the underside (in the perspective of FIG. 2) thereof. Solder bumps 104 are depicted in dashed lines in FIG. 2 since they are not visible in the perspective of FIG. 2. In the embodiment of FIGS. 1-3, there are three solder bumps 104. Solder bumps employed in embodiments of the present invention can take any suitable form but are generally circular, with a diameter of between 0.8 and 2 mm. Solder bump height can be, for example, controlled by the amount of solder paste applied to the contact pads before reflow with the typical solder bump height being in the range of 0.2 mm to 0.5 mm. The solder of the solder bumps can be any suitable solder material known to one skilled in the art and is typically an alloy of tin, lead, copper, silver, zinc and/or other metallic elements.

Analytical test strip contact pressure feature 110 is configured to operatively apply pressure (i.e., an applied force) on inserted analytical test strip TS such that at least one electrical contact (EC) of inserted analytical test strip TS is operably pressed against the solder bump(s), thereby creating a direct operable electrical connection between analytical test strip TS and the solder bump 114 of the PCB (see FIG. 3 in particular). In the embodiment of FIGS. 1-3, solder bumps 114 are disposed on a copper trace 116 of PCB 104, thus providing a signal path and/or power connection between an inserted analytical test strip and micro-controller 106 (not depicted in FIG. 3).

Analytical test strip pressure features employed in embodiments of the present invention can take any suitable form. However, the dimensions of such will generally be the same width as the associated analytical test strip and have a length sufficient to apply pressure (i.e., force) in the vicinity of the analytical test strip's electrical contacts. Moreover, although the embodiment of FIGS. 1 through 3 depicts a single analytical test strip pressure feature, a plurality of analytical test strip pressure features can be employed to provide separate pressure areas (see, for example, FIGS. 5, 6 and 8 described below). Moreover, a plurality of analytical test strip pressure features can, if desired, be beneficially configured such that each of the analytical test strip pressure features applies a force than the remainder of the analytical test strip pressure features.

Referring to FIGS. 4 through 6, another embodiment that includes a PCB 104′ with six solder bumps 114′ is depicted. In this embodiment, two pressure area PP′ (see FIGS. 5 and 6) create a slight bend (or fold) in the analytical test strip (as depicted by the longer dashed lines of FIG. 5) that bends the analytical test strip over the solder bumps and, thereby, improves the electrical contact between the solder bump and the analytical test strip electrical contacts. Predetermined variation in the shape of the analytical test strip pressure feature will provide such analytical test strip folding, with the folding typically being an offset from parallel of approximately 0.1 mm to 0.5 mm.

Referring to FIGS. 7 and 8, yet another embodiment that includes a PCB 104″ with five solder bumps 114″ is depicted. In this embodiment, three pressure areas PP″ (see FIG. 8) create as slight bends (or folds) in the analytical test strip (see FIG. 8). Predetermined variation in the shape of the analytical test strip pressure feature will provide such analytical test strip folding, with the folding typically again being an offset from parallel of approximately 0.1 mm to 0.2 mm.

Establishing an operable electrical contact between a plurality of solder bumps (on a PCB) and a plurality of electrical contacts on an analytical test strip can be challenging since small variations in height of the electrical contacts (such as carbon-based electrical contacts) and the solder bumps can leave some electrical contacts floating. However, employing multiple pressure points (as depicted in FIGS. 5-7 and 8-9) on an inserted analytical test strip to force the strip to bend over rows of solder bumps ensures that an operable electrical connection is made with the electrical contacts.

Once apprised of the present disclosure, one skilled in the art will recognize that hand-held test meter 100 is configured to operably interface with analytical test strips although without the use of conventional spring-force-based electrical contacts configured to provide an electrical connection between an analytical test strip and a PCB. Conventional strip port connectors are described in, for example, US Patent Application Publication No. US 2012-0252133 entitled “Test Meter with a Strip Port Connector Configured for Fluid Entrapment” and US Patent Application Publication No. US 2016-0205798 entitled “Watertight Casing with Integrated Electrical Contacts”, each of which is hereby incorporated in full be reference. In contrast to conventional hand-held test meters with conventional strip port connectors, embodiments of the present invention employ solder bumps of a hand-held test meter's PCB main PCB assembly to provide a direct electrical connection between electrical contacts of an inserted analytical test strip and a PCB.

FIG. 9 is a flow diagram depicting stages in a method 1000 for employing a hand-held test meter for use with an analytical test strip in the determination of an analyte (such as glucose) in a bodily fluid sample (for example, a whole blood sample). Method 1000 includes, at step 1010, inserting an analytical test strip into a housing of a hand-held test meter such that least one analytical test strip pressure feature of the housing operatively applies pressure on the inserted analytical test strip such that at least one electrical contact of the inserted analytical test strip is operably pressed against at least one solder bump of a PCB of the hand-held test meter, thereby creating direct operable electrical connection between the electrochemical-based analytical test strip and the solder bump of the PCB.

Method 1000 also includes employing the hand-held test meter to determine an analyte in a bodily fluid sample using the inserted analytical test strip received in the strip port connector (see step 1020 of FIG. 9). The received analytical test strip can be, for example, an electrochemical-based analytical test strip configured for the determination of glucose in a whole blood sample.

Once apprised of the present disclosure, one skilled in the art will recognize that methods according to embodiments of the present invention, including method 1000, can be readily modified to incorporate any of the techniques, benefits and characteristics of hand-held test meters according to embodiments of the present invention and described herein including, for example, the inclusion of a plurality of analytical test strip pressure features.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. A hand-held test meter for use with an analytical test strip for the determination of an analyte in a bodily fluid sample, the hand-held test meter comprising:
a housing the housing configured for the insertion of an analytical test strip therein;
at least one analytical test strip pressure feature; and
a printed circuit board (PCB) disposed within the housing, the PCB having at least one solder bump;
wherein the analytical test strip contact pressure feature is configured to operatively apply pressure on an inserted analytical test strip such that at least one electrical contact of the inserted analytical test strip is operably pressed against the solder bump, thereby creating a direct operable electrical connection between the analytical test strip and the solder bump of the PCB.

2. The hand-held test meter of claim 1 wherein the at least one solder bump is electrically connected to the micro-controller.

3. The hand-held test meter of claim 1 wherein the at least one analytical test strip contact pressure feature is a plurality of analytical test strip contact pressure features.

4. The hand-held test meter of claim 3 wherein the at least one solder bump is a plurality of solder bumps.

5. The hand-held test meter of claim 4 wherein the wherein the plurality of analytical test strip contact pressure features are configured to operatively apply pressure on an inserted analytical test strip such that each of a plurality of electrical contacts of the inserted analytical test strip is operably pressed against the plurality of solder bumps in a predetermined manner thereby creating a plurality of direct distinct operable electrical connections between the analytical test strip and the plurality of solder bumps of the PCB.

6. The hand-held test meter of claim 1 wherein the housing is formed of a polymeric material.

7. The hand-held test meter of claim 1 wherein the analytical test strip pressure feature is an analytical test strip pressure plate.

8. The hand-held test meter of claim 1 wherein the analytical test strip pressure feature is configured to induce at least one bend in an inserted analytical test strip.

9. The hand-held test meter of claim 8 where the at least one analytical test strip pressure feature is configured to induce a plurality of bends in an inserted analytical test strip.

10. The hand-held test meter of claim 1 wherein the at least one analytical test strip pressure feature is configured to apply a force of less than 6 Newtons against the inserted analytical test strip.

11. The hand-held test meter of claim 1 wherein the at least one analytical test strip pressure feature is formed as an integral portion of the housing;

12. The hand-held test meter of claim 1 wherein the at least one analytical test strip pressure feature is attached to, and disposed within, the housing.

13. The hand-held test meter of claim 1 wherein the analytical test strip is configured for the determination of glucose in a whole blood sample.

14. The hand-held test meter of claim 1 wherein the analytical test strip is an electrochemical-based analytical test strip.

15. A method for employing a hand-held test meter for use with an analytical test strip in the determination of an analyte in a bodily fluid sample, the method comprising:
inserting an analytical test strip into a housing of a hand-held test meter such that least one analytical test strip pressure feature of the hand-held test meter operatively applies pressure on the inserted analytical test strip such that at least one electrical contact of the inserted analytical test strip is operably pressed against at least one solder bumps of a PCB of the hand-held test meter, thereby creating direct operable electrical connection between the analytical test strip and the solder bump of the PCB.

16. The method of claim 15 wherein the analytical test strip is an electrochemical-based analytical test strip.

17. The method of claim 15 wherein the at least one solder bump is electrically connected to a micro-controller of the hand-held test meter.

18. The method of claim 15 wherein the at least one analytical test strip contact pressure feature is a plurality of analytical test strip contact pressure features.

19. The method of claim 15 wherein the at least one solder bump is a plurality of solder bumps.

20. The method of claim 19 wherein the wherein the plurality of analytical test strip contact pressure features are configured to operatively apply pressure on an inserted analytical test strip such that each of a plurality of electrical contacts of the inserted analytical test strip is operably pressed against the plurality of solder bumps in a predetermined manner thereby creating a plurality of direct distinct operable electrical connections between the analytical test strip and the plurality of solder bump of the PCB.

21. The method of claim 15 wherein the analytical test strip pressure feature is an analytical test strip pressure plate.

22. The method of claim 15 wherein the analytical test strip pressure feature is configured to induce at least one bend in an inserted analytical test strip.

23. The method of claim 15 wherein the at least one analytical test strip pressure feature is configured to apply a force of less than 6 Newtons against the inserted analytical test strip.

24. The method of claim 15 wherein the analytical test strip is configured for the determination of glucose in a whole blood sample.

25. The method of claim 15 wherein the analytical test strip is an electrochemical-based analytical test strip.
